(19) United States
(12) Patent Application Publication
(10) Pub. No.: US 2014/0323758 A1
(43) Pub. Date: Oct. 30, 2014

(54) NOVEL PROCESS FOR RACEMIZATION OF AN OPTICALLY ACTIVE (S)-3-CARBAMOYLMETHYL-5-METHYL-HEXANOIC ACID TO CORRESPONDING 3-CARBAMOYLMETHYL-5-METHYL-HEXANOIC ACID RACEMATE

(71) Applicant: LUPIN LIMITED, Mumbai, Maharashtra (IN)

(72) Inventors: Nikhil Shashikant Bhatt, Ankaleshwar (IN); Amit Yagneshkumar Trivedi, Ankaleshwar (IN); Dipak Jayantilal Gandhi, Ankaleshwar (IN); Bhikhulal Mohanbhai Kachhadia, Ankaleshwar (IN); Vijay Basantilal Shah, Ankaleshwar (IN); Yogesh Dhirubhai Finaviya, Ankaleshwar (IN)

(21) Appl. No.: 14/360,451

(22) PCT Filed: Nov. 16, 2012
(86) PCT No.: PCT/IB2012/056474

§ 371 Date: May 23, 2014

(30) Foreign Application Priority Data
Nov. 24, 2011 (IN) 1493/KOL/2011

(51) Int. Cl. C07C 227/36

(52) U.S. Cl. CPC C07C 227/36
USPC 562/401

(57) ABSTRACT

A novel process for racemization of (S)-3-carbamoylmethyl-5-methyl-hexanoic acid to 3-carbamoylmethyl-5-methyl-hexanoic acid racemate has been developed.

### FIELD OF THE INVENTION

This invention relates to process for racemization of (S)-3-carbamoyl -methyl-5-methyl-hexanoic acid (I) to 3-carbamoylmethyl-5-methyl-hexanoic acid racemate (III).

### BACKGROUND OF THE INVENTION

Pregabalin, (S)-3-(aminomethyl)-5-methylhexanoic acid, a compound having the following chemical structure of formula

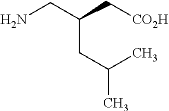

Pregabalin is also known as γ-amino butyric acid or (S)-3-isobutyl GABA. Pregabalin, marketed under the name Lyrica®, has been found to activate GAD (L-glutamic acid decarboxylase). Pregabalin has a dose dependent protective effect on-seizure, and is a CNS-active compound. Pregabalin is useful in anticonvulsant therapy, due to its activation of GAD, promoting the production of GABA, one of the brain's major inhibitory neurotransmitters, which is released at 30 percent of the brain synapses. Pregabalin has analgesic, anti-convulsant, and anxiolytic activity.

Pregabalin was first reported in U.S. Pat. No. 6,197,819 along with its chiral syntheses. The process for preparing Pregabalin was reported in U.S. Pat. No. 5,616,793, which describes the resolution of (R)-3-(aminomethyl)-5-methylhexanoic acid using (R)-(+)-α-phenylethylamine. The isolated above isomer was reacted with an alkali hydroxide, and bromine. This reaction mass was heated to 80° C. and the Pregabalin was isolated. A disadvantage of this process is the yield is inferior due to use of a single isomer.

Chavan and coworkers in Organic Process Research and Development, 2009, 13, 812-814, have worked to develop a process for racemization of (S) isomer of carbamoylmethyl-5-methyl-hexanoic acid. This process was emphasized on the use of various bases and further conversion to racemized product. The disadvantage of this process is use of base and inferior in case of yield also.

Although, in the said publication, racemization of 3-carbamoylmethyl-5-methylhexanoic acid substrate has been reported but there is no report for the racemization of substrate having superior yield and avoidance of harmful chemicals.

This invention provides a method for racemization of optically active (S)-3-carbamoylmethyl-5-methyl-hexanoic acid (I) to 3-carbamoylmethyl-5-methyl-hexanoic acid racemate (III).

### OBJECT OF THE INVENTION

The object of the present invention is to provide a process for recycling of (S)-3-carbamoylmethyl-5-methyl-hexanoic acid (I) via converting into corresponding imide i.e. 3-isobutyl glutarimide (II), followed by racemization to obtain 3-carbamoylmethyl-5-methyl-hexanoic acid racemate (III), which could be reused for resolution through diastereomeric salt formation with cinchonidine thereby improving the atom economy and hence further reduce the cost for the synthesis of pregabalin.

### SUMMARY OF INVENTION

The present invention is directed towards racemization of (S)-3-carbamoylmethyl-5-methyl-hexanoic acid (I) to 3-carbamoylmethyl-5-methyl-hexanoic acid (III) with heating at high temperature such as 60 to 140° C. or optionally with a base and optionally in a solvent or mixtures thereof. The invention is summarized below scheme.

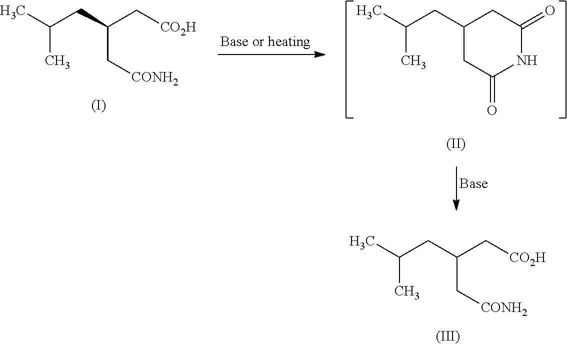

### DETAILED DESCRIPTION OF THE INVENTION

The present invention relates to a process for racemization of an isomer i.e. (S)-3-carbamoylmethyl-5-methyl-hexanoic acid. This isomer is formed as a by-product in the process for preparation of pregabalin. The conversion of the formed isomer in to its racemic form.

This undesired isomer of 3-carbamoylmethyl-5-methyl-hexanoic acid is in large amount in mother liquor and ultimately affects on the yield of pregabalin. Hence the present invention provides a process of racemization of undesired isomer of 3-carbamoylmethyl-5-methyl-hexanoic acid i.e. (S)-3-carbamoylmethyl-5-methyl-hexanoic acid.

The present process of racemization of undesired isomer is carried out by converting to a cyclic amide and then racemate using base hydrolysis.

The S-isomer of 3-carbamoylmethyl-5-methyl-hexanoic acid was converted to a cyclic amide in situ and then hydrolyzed with an alkali hydroxide to produce 3-carbamoylmethyl-5-methyl-hexanoic acid racemate as depicted below scheme 3.

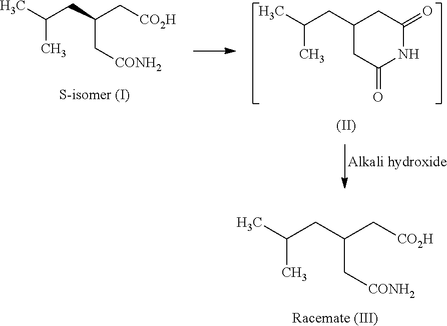

The present process of racemization of (S)-3-carbamoylmethyl-5-methyl-hexanoic acid involves heating of (S)-3-carbamoylmethyl-5-methyl-hexanoic acid at higher temperature for about 2 to 4 hours to produce a cyclic imide compound, which upon hydrolysis with an alkali hydroxide solution gives the alkali salt of 3-carbamoylmethyl-5-methyl-hexanoic acid racemate. This is on acid treatment gives 3-carbamoylmethyl-5-methyl-hexanoic acid racemate.

The temperature used for this reaction is selected from 60 to 140° C. Preferably, the temperature used for reaction is 130 to 140° C.

This reaction is not necessarily emphasized on the use of solvent. But the solvents, which are useful for racemization reaction is selected from water, methanol, ethanol. The preferred solvent is water.

The alkali hydroxide used for racemization reaction is selected from sodium hydroxide or potassium hydroxide. The preferred alkali hydroxide is sodium hydroxide.

The acid used for racemization reaction is selected from sulfuric acid or hydrochloric acid. The preferred acid is sulfuric acid.

The crude 3-carbamoylmethyl-5-methyl-hexanoic acid racemate is not necessarily emphasized on the purification process. But the purification process is useful in chloroform or dichloromethane, preferably in chloroform.

Thus, this invention provides a process of racemization of (S)-3-carbamoylmethyl -5-methyl-hexanoic acid (I) comprising of:
a) (S)-3-carbamoylmethyl-5-methylhexanoic acid heating or optionally treating with a base at higher temperature;
b) treating the solution with an alkali or alkali hydroxide and further acidifying the solution with an acid to obtain (3)-carbamoylmethyl-5-methyl -hexanoic acid racemate (III) and;
c) isolating the (3)-carbamoylmethyl-5-methyl-hexanoic acid racemate (III).

The present invention relates the reuse of the available and potential isomer to the racemate compound, which necessarily improves the yield and reduces the cost of pregabalin.

The present invention also avoids the use of hazardous base and solvent to prepare the pregabalin in green, cost effective and environment friendly manner.

The example below is intended to illustrate specific embodiment of the invention and is not intended to limit the scope of the specification, including the claims, in any manner.

### EXAMPLE

### Racemization of (S)-3-Carbamoylmethyl-5-Methyl-Hexanoic Acid

A reactor equipped with overheard stirring is charged with 50 gm of dry (S)-3-carbamoylmethyl-5-methyl-hexanoic acid (71.5 gm of (S)-3-carbamoylmethyl-5-methyl-hexanoic acid, if wet) and was heated at 60 to 80° C. The temperature of reaction mass was increased to 130 to 140° C. and was maintained for 2 hours. The reaction mass was cooled to 90° C. and 20% sodium hydroxide solution was charged at 50 to 60° C. The reaction mass was stirred for 15 to 30 minutes. After stirring, the reaction mass was cooled to 30 to 35° C. and was filtered through celite bed to remove particles. The obtained solution was acidified to pH 2.5 to 3 using sulfuric acid and was stirred for 15 to 30 minutes for half an hour. The crude product was filtered and dried. The 44 to 46 gm of 3-carbamoylmethyl-5-methyl-hexanoic acid racemate was obtained. The achieved product was purified by chloroform.

1) A process of racemization of (S)-3-carbamoylmethyl-5-methyl-hexanoic acid (I) comprising of:
a) (S)-3-carbamoylmethyl-5-methyl-hexanoic acid heating or optionally treating with a base at higher temperature;
b) treating the solution with an alkali or alkali hydroxide and further acidifying the solution with an acid to obtain (3)-carbamoylmethyl-5-methyl-hexanoic acid racemate (III) and;
c) isolating the (3)-carbamoylmethyl-5-methyl-hexanoic acid racemate (III).

2) The process as claimed in claim 1, wherein step (a) the heating temperature is 60 to 140° C.

3) The process as claimed in claim 2, the heating temperature is 60 to 80° C.

4) The process as claimed in claim 1, wherein step (a) the optionally base is selected from pyridine, piperidine, sodium hydroxide, and potassium hydroxide.

5) The process as claimed in claim 1, wherein step (b) the alkali or alkali hydroxide is selected from sodium hydroxide or potassium hydroxide.

6) The process as claimed in claim 5, the preferred alkali hydroxide is sodium hydroxide.

7) The process as claimed in claim 1, wherein step (a) the acid is selected from carboxylic acid or mineral acid.

8) The process claimed in claim 7, wherein the acid is selected from hydrochloric acid or sulfuric acid.

9) The process claimed in claim 8, wherein the preferred acid is sulfuric acid.
